(19) United States
(12) Patent Application Publication
(10) Pub. No.: US 2025/0237639 A1
(43) Pub. Date: Jul. 24, 2025

(54) BLOOD LEAD TESTING SYSTEM AND METHODS THEREOF

(71) Applicant: Meridian Bioscience, Inc., Cincinnati, OH (US)

(72) Inventors: David Beliveau-Viel, North Chelmsford, MA (US); Mahadurage Sachintha Wijesinghe, Westford, MA (US); Ahmad H. Aljuboori, Methuen, MA (US); Tyler Jeffrey Kays, Edison, NJ (US)

(21) Appl. No.: 19/032,719

(22) Filed: Jan. 21, 2025

Related U.S. Application Data
(60) Provisional application No. 63/623,489, filed on Jan. 22, 2024.

(51) Int. Cl. G01N 33/487; B01L 3/00; G01N 27/327; G01N 27/42; G01N 33/49

(52) U.S. Cl. CPC G01N 33/48714; B01L 3/502; G01N 27/327; G01N 27/423; G01N 33/49; B01L 2300/0645; B01L 2300/0816

(57) ABSTRACT

Systems and method for use in measuring lead levels in biological samples is provided. Using square wave coulometry and colloidal metallic particles impregnated on printed carbon electrodes on a sensor, the system provides a rapid, reliable, portable and inexpensive means of detecting low lead levels.

### CROSS REFERENCE TO RELATED APPLICATIONS

This application claims the benefit of U.S. application Ser. No. 63/623,489, entitled BLOOD LEAD TESTING SYSTEM AND METHODS THEREOF, filed on Jan. 22, 2024, the disclosure of which is incorporated herein by reference it its entirety.

### BACKGROUND

Heavy metals such as lead have received increasing recognition as serious threats to the environment and to human health. The effects of lead may not be acute, as chronic toxicity is of particular concern because this metal accumulates in tissues over a period of long-term exposure. This may lead to mental and physical abnormalities, especially in the very young. Blood lead testing systems are used clinically and for population surveillance to measure exposure to environmental lead which can have toxic effects. Samples of whole blood (typically capillary or venous samples) are typically analyzed by lab equipment and test kits that employ electrochemical or spectroscopic methods to quantify lead content, with results like μg of lead per dL of blood. Portable, point-of-care lead testing systems provide rapid analysis from a small blood sample at near patient settings, while high-throughput blood lead testing systems are used by clinical diagnostic laboratories, often utilizing blood lead analyzers connected to central laboratory automation systems for efficiency in handling high sample volumes. By quantifying blood lead levels, medical and public health interventions can be taken if dangerous exposures or poisonings are revealed.

### BRIEF DESCRIPTION OF THE DRAWINGS

The present disclosure will be more readily understood from a detailed description of some example embodiments taken in conjunction with the following figure:

FIG. 1 depicts a blood lead testing system in accordance with one non-limiting embodiment.

FIG. 2 depicts an example embodiment of an electrochemical stripping sensor in accordance with one non-limiting embodiment.

FIG. 3 depicts an exploded view of the electrochemical stripping sensor of FIG. 2.

FIG. 4 depicts a schematic representation of a circuit of an electrochemical analyzer in accordance with one non-limiting embodiment.

FIG. 5 depicts an example of an electrochemical stripping assay, test or measurement sequence as may be performed by a microprocessor based electrochemical stripping analyzer in accordance with one non-limiting embodiment.

FIG. 6 depicts a current flowing through a working electrode during an example electrochemical measurement sequence in accordance with one non-limiting embodiment.

FIG. 7 depicts a computation of a square wave coulometric (SWC) value, result or stripping signal from an electrochemical measurement sequence such as may be performed by a microprocessor based electrochemical analyzer in accordance with one non-limiting embodiment.

### DETAILED DESCRIPTION

Various non-limiting embodiments of the present disclosure will now be described to provide an overall understanding of the principles of the structure, function, and use of the systems, apparatuses, devices, and methods disclosed. Those of ordinary skill in the art will understand that systems, apparatuses, devices, and methods specifically described herein are non-limiting embodiments. The features illustrated or described in connection with one non-limiting embodiment may be combined with the features of other non-limiting embodiments. Such modifications and variations are intended to be included within the scope of the present disclosure.

The systems, apparatuses, devices, and methods disclosed herein are described in detail by way of examples and with reference to the figures. The examples discussed herein are examples only and are provided to assist in the explanation of the apparatuses, devices, systems and methods described herein. None of the features or components discussed below should be taken as mandatory for any specific implementation of any of these apparatuses, devices, systems or methods unless specifically designated as mandatory. For ease of reading and clarity, certain components, modules, or methods may be described solely in connection with a specific embodiment. In this disclosure, any identification of specific techniques, arrangements, etc. are either related to a specific example presented or are merely a general description of such a technique, arrangement, etc. Identifications of specific details or examples are not intended to be, and should not be, construed as mandatory or limiting unless specifically designated as such. Any failure to specifically describe a combination or sub-combination of components should not be understood as an indication that any combination or sub-combination is not possible. It will be appreciated that modifications to disclosed and described examples, arrangements, configurations, components, elements, apparatuses, devices, systems, methods, etc. can be made and may be desired for a specific application. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

Throughout this disclosure, references to components or modules generally refer to items that logically can be grouped together to perform a function or group of related functions. Like reference numerals are generally intended to refer to the same or similar components. Components and modules can be implemented in software, hardware, or a combination of software and hardware. The term “software” is used expansively to include not only executable code, for example machine-executable or machine-interpretable instructions, but also data structures, data stores and computing instructions stored in any suitable electronic format, including firmware, and embedded software. Those of ordinary skill in the art will recognize that the systems, apparatuses, devices, and methods described herein can be applied to, or easily modified for use with, other types of equipment, can use other arrangements of computing systems, and can use other protocols, or operate at other layers in communication protocol stacks, than are described.

A potentiostat is an electrochemistry instrument capable of controlling an electrical potential applied to a solution and measuring a resulting current flowing through it. Potentiostats used in analytical chemistry typically comprise three electrodes: one working electrode (WE), where the chemical reaction of interest is occurring, one reference electrode (RE), against which the WE potential is measured (WE-RE), and one counter electrode (CE), which adopts whatever potential, and supplies whatever current, are necessary to maintain the WE-RE potential difference at the commanded value. To precisely measure the current flowing through the WE, the potentiostat may utilize a transimpedance amplifier (TIA), which maintains the WE at a potential of 0 V by sourcing or sinking any charge present at the WE that would otherwise change its potential. The TIA flows this source and sink current through a resistor of precisely known value, causing a voltage drop whose measurement provides a precise indication of the WE current.

Anodic stripping voltammetry (ASV) is an electrochemical technique enabling the detection and quantification of metals ions in aqueous solutions. Typically, the solution under test is placed in a three-electrode electrochemical cell. A potentiostat first applies an oxidizing potential to clean the WE, followed by a reducing potential that causes the metal ions in solution to plate and concentrate on the WE. The potential is then increased gradually towards a more oxidizing value, anodically stripping the plated metal analyte from the WE. The WE current is measured as a function of the varying potential during this ramp; each metal species will leave the electrode at its characteristic oxidation potential and produce an electrical current peak as it does so. Each peak's area is then computed and correlated with values obtained using calibration solutions to provide a quantitative measurement of the concentration of analyte in the solution under test.

Square Wave Voltammetry (SWV) is a technique whereby the potential is varied in pulses instead of continuous potential ramps. Specifically, in the case of an increasing potential sweep, the potential is increased by some amount (x+s), and after a delay t a current measurement i₁ is made (forward current), following which the potential is decreased by a lesser amount x, and after the same delay t a second current measurement i₂ is made (reverse current). The cycle then repeats, with two current measurements made every cycle, while the potential varies like a staircase ramp of step s superimposed to a square wave of amplitude x/2. Critically, the delay t before each current measurement is chosen so that the capacitive current due to the step change in potential, has mostly decayed, while the Faradaic current due to the reaction of the analyte on the electrode is still present, thus helping suppress capacitive background currents. Further, subtracting the reverse current measurements i₂ from the forward current i₁ can provide baseline correction. This technique is readily applicable to the stripping step in ASV.

Disclosed in the present application are a sensor, analyzer, system, and methods for analyzing a sample for at least one analyte. In some embodiments, the sample is a vertebrate or mammalian blood sample, and the sample is placed on the sensor of the present disclosure, the sensor being readable using the analyzer. The sensor can be an electrochemical sensor, which can also be referred to as a strip, microfluidic chip, consumable, or disposable. In some embodiments, the sample can be treated with a reagent to facilitate the analysis. In some embodiments, the reagent is an acid such as hydrochloric acid. In an example embodiment, an electrochemical stripping sensor that employs SWC is used for the measurement of blood-lead. In some embodiments, conducting inks are printed on a supporting material to fabricate reference, counter and a plurality of working electrodes, such as during a screen-printed process. In some embodiments, colloidal gold sol mixed with a cationic polymer is deposited on each of the working electrodes for providing better sensitivity to lead in sample solution by techniques such as ink-jet printing, volumetric deposition, and air brush spraying. Sensors such as, for example, screen-printed electrodes that are used to detect low blood lead concentrations, are susceptible to falsely elevated lead readings due, for example, to trace amounts of lead being present in the sensor materials due to naturally occurring background levels in the environment. For example, the carbon used to make the carbon ink of a screen-printed electrode may contain minute particles of lead. One area of such sensors that is particularly sensitive to lead contamination is the working electrode surface, which is where the lead present in the blood sample is deposited and then measured during the stripping assay.

Colloidal gold sols are easily deposited through well-developed liquid handling techniques. The electrochemical activity relative to mass is greater than for bulk gold. The lead sensitivity of colloidal gold modified carbon electrodes is greater than the lead sensitivity of bulk gold electrodes/U.S. Pat. Nos. 5,334,296; 5,391,272; 5,217,594; and 5,368,707 provide additional description regarding example approaches for preparing colloidal gold electrodes, each of which is incorporated herein by reference in its entirety.

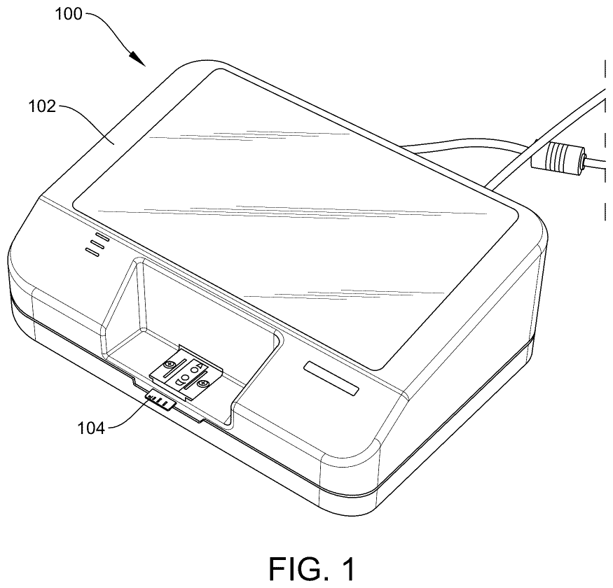

In some embodiments, blood lead testing systems in accordance with the present disclosure can use anodic stripping voltammetry (ASV) to quantitatively measure, in approximately 3 minutes, the amount of lead in a sample of human capillary whole blood. Referring to FIG. 1, a blood lead testing system 100 can include two main interacting subsystems: an analyzer 102, i.e. an electronic instrument containing a custom potentiostat implementing square wave voltammetry (SWV) techniques to perform ASV, and a sensor 104, i.e. a disposable test strip comprising screen-printed electrodes and a fluidic channel, into which is dispensed the diluted capillary whole blood sample. FIG. 1 depicts the sensor 104 inserted into the analyzer 102. The system can further comprise a glass capillary tube calibrated to collect, for example, 50 μL of capillary whole blood, a tube of treatment reagent (TR), and a disposable transfer pipette.

In one example workflow, an operator follows the analyzer's 102 on-screen guidance to collect 50 μL of capillary whole blood from the patient into the provided glass capillary and uses the provided plunger to expel the collected blood into an open TR tube. The operator then caps the tube and mixes by inversion.

The operator picks a sensor 104 from a desiccated vial and inserts it into the analyzer 102, which then prompts to open the TR tube cap and dispense a few drops of diluted sample onto the sensor 104 with the disposable transfer pipette.

The analyzer 102 automatically detects that the preparation adequately flowed into the sensor's fluidic channel and starts the electrochemical measurement. After approximately 3 minutes, a quantitative blood lead concentration result is provided on the screen and stored in the analyzer's database for output to a USB thumb drive, a laboratory information system (LIS) or an optional label printer.

Blood lead testing systems in accordance with the present disclosure can be provided in kits, comprising an analyzer and sensors, which are available to health professionals, ranging from individual clinics and test sites to moderate-throughput laboratories.

In some embodiments, the kit can comprise, for example, an analyzer, a handheld 2D barcode reader, required cables and illustrated operating instructions. A test kit can comprise sensors and consumables for 48 tests, as well as instructions for use.

In one example workflow, an operator follows the analyzer's on-screen guidance to collect 50 μL of capillary whole blood from the patient into the provided glass capillary and uses the provided plunger to expel the collected blood into an open TR tube. The operator then caps the tube and mixes by inversion.

The operator picks a sensor from a desiccated vial and inserts it into the analyzer, which then prompts to open the TR tube cap and dispense a few drops of diluted sample onto the sensor with the disposable transfer pipette.

The analyzer automatically detects that the preparation adequately flowed into the sensor's fluidic channel and starts the electrochemical measurement. After approximately 3 minutes, a quantitative blood lead concentration result is provided on the screen and stored in the analyzer's database for output to a USB thumb drive, a laboratory information system (LIS) or an optional label printer.

Blood lead testing systems in accordance with the present disclosure can be provided in kits, comprising an analyzer and sensors, which are available to health professionals, ranging from individual clinics and test sites to moderate-throughput laboratories.

In some embodiments, the kit can comprise, for example, an analyzer, a handheld 2D barcode reader, required cables and illustrated operating instructions. A test kit can comprise sensors and consumables for 48 tests, as well as instructions for use.

In an example patient test workflow, the analyzer is placed on a table, connected to a wall outlet or rechargeable battery pack, and powered on. After a short instrument start up and self-test sequence, the operator enters their login credentials using the analyzer's touchscreen and accesses the main menu.

The operator selects the patient test workflow and, optionally, enters the patient's identifier manually or with the 2D barcode scanner. The operator then follows the analyzer's on-screen guidance to clean the patient's hands and puncture site, prick the patient's finger with a lancet and collect 50 μL of capillary whole blood into a calibrated glass capillary provided in a test kit.

The operator then uses the provided plunger to expel the collected blood from the capillary into an open TR tube, capping the tube and inverting it approximately 10 times to dilute the specimen.

The operator picks a sensor from a provided desiccated vial and inserts it into the analyzer, which detects its lot number and fetches the corresponding lot-specific calibration information from its database. If the lot is not expired and no issues are detected, the operator then follows the analyzer's prompt to open the TR tube cap and dispense a few drops of diluted sample onto the sensor with the provided disposable transfer pipette.

Upon detecting that the diluted sample adequately flowed into the sensor's fluidic channel and that all electrodes are properly wetted, the analyzer automatically starts the electrochemical measurement. After approximately 3 minutes, if no error is detected, a numerical blood lead concentration result is provided on the screen and stored in the analyzer's database along with the patient identifier and other relevant information. The sensor and materials are safely discarded.

The operator can output the result to a USB thumb drive, a laboratory information system (LIS) or an optional label printer, or other output means, and return to the main menu to conduct another test.

Results can also be reviewed and exported in batch from each analyzer by operators and site supervisors, who can use the recorded patient identifiers to associate each patient's blood lead result with the personally identifiable information stored in the site's secure systems and fulfil their state's reporting requirements.

With regard to an example quality control workflow, the test kit can contain one vial each of Level 1 and Level 2 quality control solutions which, when tested as if they were blood, produce lead results at the bottom and the top of the measurement range, respectively. These solutions are room temperature stable and can be used with the analyzer's quality control workflow to check and document that the system is in good working order and the operators are proficient.

In some embodiments, the analyzer can require that all registered operators successfully run Level 1 and Level 2 quality controls at least once a month, or on other suitable timetables.

Blood lead testing systems in accordance with the present disclosure can use ASV to quantitatively measure, in approximately 3 minutes, the amount of lead (Pb) in a sample of human capillary whole blood. ASV has been used for decades to detect lead contamination in many materials, including whole human blood. While ICP-MS is typically used for the quantification of blood lead, ASV remains widely used at the point of care thanks to inexpensive screen-printed electrodes and instrumentation, for which an instrumental limit of detection of 1.4 μg/dL has been reported.

ASV is an electrochemical technique comprising three steps: (1) electrode conditioning, where oxidizing impurities are removed from the working electrode surface; (2) metal deposition, where a potential is applied to reduce metal ions in the positive oxidation state onto the working electrode as metal in the 0 state [e.g., Pb²⁺+2e⁻→Pb]; and (3) metal stripping, where the working electrode potential is increased towards more positive values, oxidizing each deposited species back to its ion [e.g., Pb→Pb²⁺] as its redox potential is reached. As this occurs, a current peak is measured whose intensity correlates with the species' concentration in the analytical solution. Square Wave Voltammetry (SWV) and Square Wave Coulometry (SWC) are applied to effectively remove the contribution of non-Faradic current and increase the signal-to-background ratio.

While each species' redox potential can be derived theoretically from the Nernst equation, in practice it greatly depends on the working electrode material. Usually, peak potentials and calibration curves are determined experimentally for a given system configuration. Theoretical equations, however, highlight parameters that must be controlled for an accurate result, such as temperature, duration, viscosity and agitation.

A blood lead testing systems described herein may satisfy the following performance metrics:
A limit of detection (LoD) of 1.5 μg/dL;
A reportable range spanning from the LoD to 65 μg/dL.

One key of the blood lead testing system's ability to detect low levels of lead in whole blood reliably and accurately at the point of care rests in its multiple features designed to mitigate sources of error at the pre-analytical, analytical and post-analytical phases.

As the operator proceeds through the patient test workflow, as described above, multiple design features can intervene to mitigate measurement error before, during, and after the actual analysis.

As the blood lead testing system is powered on, its advanced self-check directly verifies that all sensing circuit components are effective across the blood lead reportable range. The analyzer prevents operators who missed their quality control (i.e., as may be required monthly) from initiating a patient test, helping ensure proficiency. On-screen illustrated instructions guide operators from cleaning the puncture site to starting the measurement.

The supplied calibrated glass capillary tube is coated with anticoagulant to prevent blood clotting before dilution.

The TR tube is supplied with an accurately measured quantity of diluent, which is essential to obtain a reproducible dilution ratio. Its materials allow visual confirmation of the mixing.

The TR itself, which is dilute HCl, is room temperature stable and effectively lyses the red blood cells, in which 99% of blood lead is found, denatures bound proteins and solubilizes Pb²⁺ ions in an electrolyte suitable for electrochemical measurement.

The analyzer's internal barcode scanner can automatically retrieve the inserted sensor's lot and serial numbers to ensure it is not expired and has not been used before. The software can fetch the lot's calibration data from memory, or the operator can retrieve it by scanning a barcode on the sensor vial, which ensures the analyzer is correctly calibrated.

The sensor's transparent, hydrophilic fluidic channel mitigates incomplete electrode coverage and external contamination which could introduce variability in the results.

The analyzer can detect the change in conductivity and automatically starts the electrochemical assay, which it conducts with the necessary precise timing. To curb device complexity and variability, no solution agitation occurs during deposition. Deposition current and sensor temperature are monitored throughout the deposition step to precisely compensate for their effects. In some embodiments, temperature control of the sensor can be utilized to increase its signal.

Two or more large carbon working electrodes can each be covered with a layer of colloidal gold in a cationic polymer. The former provides nucleation sites for Pb reduction with larger total area than bulk gold, while the latter stabilizes the colloid and helps block the diffusion of interfering species.

The ASV scan is performed using SWV to effectively suppress capacitive currents. The lead current peak from each working electrodes can be processed independently. Both measurements are averaged to statistically decrease variability, unless a discrepancy between the two values, or any other condition, indicates an invalid result. Repeat ASV scans help confirm the result. The signal can be corrected for temperature effects and converted to a lead concentration using the lot's calibration data, which also compensates for any within-lot trend detected at the factory. The result can be stored in the analyzer's database for later review, and can be printed, exported to external storage and sent via LIS to mitigate transcription errors and ensure traceability.

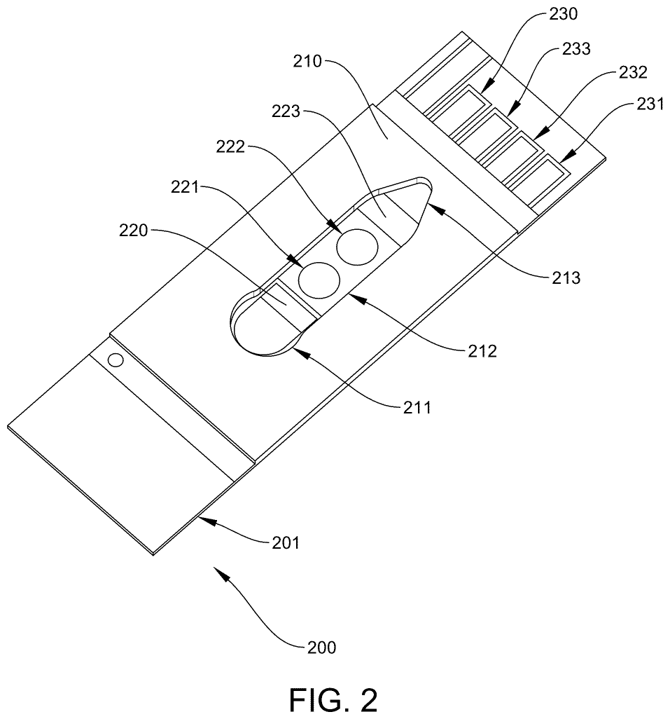

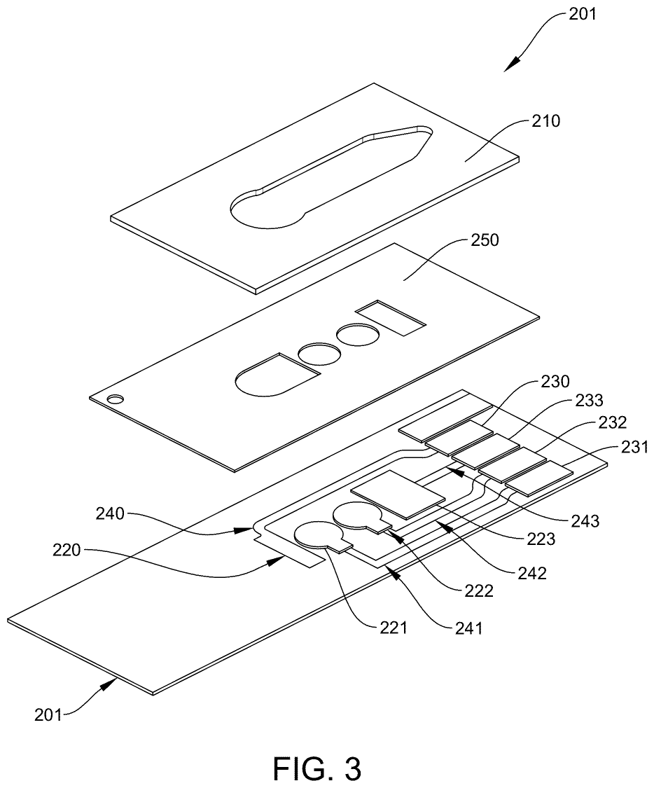

FIG. 2 depicts an example embodiment of an electrochemical stripping sensor 200 according to the present disclosure. FIG. 3 depicts an exploded view of the electrochemical stripping sensor 200. Referring to FIGS. 2-3, the sensor 200 can be generally rectangular in shape and having a body that comprises a base layer 201 and a channel layer 210 disposed on, or glued, welded or bonded to a base layer 201. The base layer 201 can be, for example, a sheet of a polymer of a thickness between 10 and 1000 microns or more. The channel layer 210 can at least partially define a fluidic channel 212 extending between a first or proximal end 211 and a second or distal end 213. The fluidic channel 212 can be generally rectilinear as illustrated in FIG. 2, but can be of any shape or combination of shapes without departing from the present disclosure.

The channel layer 210 can provide a lid, upper surface, or roof (not shown) to the fluidic channel 212 enclosing the volume of fluidic channel 212 between the lid and base layer 201, with the volume being function of a thickness of channel layer 210. The lid can be provided, for example, by a thin layer of polymer material glued, welded or bonded to channel layer 212 and forming its topmost surface. In some embodiments, the lid can be provided by a molded feature of channel layer 212. The channel layer 210 can further provide a sample inlet port at first end 211 defined by an opening or hole in the lid, upper layer or roof of channel 112, enabling the ingress of fluid sample into channel 212. The channel layer 210 can further provide a channel vent hole at second end 213 defined by an opening or hole in the lid, upper layer or roof of the channel 212, enabling egress of trapped air and adequate filling of the channel 212 with sample.

The base layer 201 can provide a reference electrode 220, a first working electrode 221, a second working electrode 222, a counter electrode 223, or any number or combination thereof. The electrodes can be provided in such a manner as to or partially or totally be wetted by or come in contact with a liquid sample inserted in a channel 212. The base layer 201 can further provide, for example by way of traces of conducting ink, an electrical connection 240 between a reference electrode 220 and a reference electrode contact 230, an electrical connection 241 between a first working electrode 221 and a first working electrode contact 231, an electrical connection 242 between a second working electrode 222 and a second working electrode contact 232, and an electrical connection 243 between a counter electrode 223 and a counter electrode contact 233, the contacts being disposed on a base layer 201 so as to make electrical connection with an electrochemical analyzer provided with, for example, corresponding spring-loaded metallic contacts.

In one embodiment, the reference electrode 220 can include in a layer of conductive silver ink, a layer of silver chloride ink, a layer of ink containing a mixture of silver and silver chloride, a layer of silver ink forming a layer of silver chloride upon curing or exposure to reagents such as dilute hydrochloric acid, or any number, mixture, arrangement or combination thereof.

In one embodiment, the counter electrode 223 can include in a layer of conductive silver or platinum or metallic ink, a layer of ink containing carbon particles, a layer of glassy carbon particles or nanoparticles, a layer of cationic polymer, a layer of gold, platinum or metallic colloid, or any number, mixture, arrangement or combination thereof. In one embodiment, the first working electrode 221 can include in a layer of conductive silver or metallic ink, a layer of ink containing carbon particles, a layer of glassy carbon particles or nanoparticles, a layer of cationic polymer, a layer of gold, platinum or metallic colloid, or any number, mixture, arrangement or combination thereof. In one embodiment, the second working electrode 222 can include in a layer of conductive silver or metallic ink, a layer of ink containing carbon particles, a layer of glassy carbon particles or nanoparticles, a layer of cationic polymer, a layer of gold, platinum or metallic colloid, or any number, mixture, arrangement or combination thereof. In one embodiment, the second working electrode 222 is of essentially identical shape, dimension and constitution as a first working electrode 221. In one embodiment, a second working electrode 222 is of different shape, dimension or constitution than a first working electrode 221. Further, any number of working electrodes in any combination of shapes, dimensions or constitutions can be provided without departing from the scope of the invention.

Base layer 201 can further provide a layer of masking ink 250 which can be substantially electrically insulating and electrochemically inert. The masking ink can prevent contact between the liquid sample and selected areas of conductive inks and materials present on base layer 201 where such contact would be undesirable, for example because the contact would cause uncontrolled chemical reactions, uncontrolled electrical current flow, short-circuits, degradation of materials, functionality or performance, or a combination thereof. The masking ink 250 can also be utilized in precisely and reproducibly defining the active areas of reference electrode 220, working electrodes 221 and 222 or counter electrode 223, the active areas being areas where the electrodes contact a fluid sample. The masking ink 250 can further aid in manufacturing processes by providing mechanical and chemical protection to inks applied to base layer 201, containing reagents such as colloidal gold during their deposition onto working electrodes 221 and 222, and defining visual reference points utilized by operators and machinery.

In accordance with the present disclosure, the inks, polymers, metals, colloids or mixtures thereof can be applied to base layer 201 using methods such as screen-printing, silk screening, stamping, rolling, UV curing, heat curing or drying, vapor deposition, plating, line or pattern spraying, inkjet printing, liquid drop deposition, or any applicable manufacturing, fabrication or assembly method or process in the art, or any sequence or combination thereof. Further, any shape, dimension, or position of electrodes or other features of base layer 201 or other aspects of the invention can be constrained by, or optimally selected to facilitate, the manufacturing process.

With regard to an example screen printing process for depositing electrodes onto base layer 201, the process can employ a mesh screen or stencil with a defined pattern through which conductive inks are pressed using a squeegee or similar implement. The conductive inks can comprise carbon, silver, silver/silver chloride, platinum, gold, or other conductive materials suspended in a suitable carrier medium. The mesh screen's characteristics, including mesh count, thread diameter, and emulsion thickness, can be selected to achieve desired electrode thickness and definition. Multiple screen-printing passes can be performed to build up electrode thickness or to create layered structures. The screen-printed electrodes can be dried or cured using heat treatment, typically at temperatures between 80° C. and 150° C., though specific temperatures and durations depend on the ink composition and desired properties. Post-printing treatments can be applied to enhance electrode performance or modify surface characteristics. Such treatments can include plasma cleaning, chemical modification, or application of additional layers such as membranes or metallic colloids. When applying metallic colloids, the colloid solution can be deposited onto specific electrode surfaces through controlled dispensing methods such as pipetting or automated liquid handling systems. The resulting modified electrodes can exhibit enhanced sensitivity, selectivity, or other desirable electrochemical properties.

In some embodiments, the reference electrode 220 is provided that comprises a layer of conductive silver ink which can have the ability to form a layer of silver chloride upon exposure to reagents such as dilute hydrochloric acid; the first working electrode 221 is provided that comprises of a layer of carbon conductive ink onto which is applied a layer of gold colloid suspended in a cationic polymer; the second working electrode 222 is provided of essentially similar shape, dimension and composition as the first working electrode 221; and the counter electrode 223 is provided comprising a layer of silver conductive ink onto which is applied a layer of carbon conductive ink; traces of silver ink between 25 and 2000 microns wide provide electrical connection between each electrode and its respective electrode contact.

In some embodiments, the layer of masking ink 250 defines an approximately circular active area for each working electrode, the circular active area being of a size, shape or position selected to facilitate a liquid drop deposition process of gold colloid solution. In some embodiments, the facilitation can result from matching the size of an active area with the size of a deposited gold colloid solution drop to ensure a complete coverage of the active area with a gold colloid solution, prevent spreading of a gold colloid solution drop beyond the active area prior to its drying, or attain any other advantage or combination thereof. In some embodiments, working electrodes each have an approximately circular active area having a diameter between about 1 and about 20 millimeters, such as a diameter of approximately 3, 4, 5, 6 or 7 millimeters. In some embodiments, the facilitation can result from positioning 2, 3, 4 or more working electrodes in a linear, circular, rectangular, triangular, or other pattern or combination thereof, onto base layer 201. In some embodiments, working electrodes each have an approximately rectangular active area having a width between about 0.1 and about 10 millimeters or more and a length between about 1 and about 50 millimeters or more.

In some embodiments, an electrochemical sensor 200 provides a human-readable or machine-readable marking, barcode, two-dimensional (2D) barcode, quick-response (QR) code, datamatrix, radio frequency (RF) identification, or any number or combination thereof. In some embodiments, the marking is provided on a base layer 201, channel layer 210, masking ink layer 250 or any other element or combination thereof, by way of laser engraving, laser marking, laser ablation, inkjet printing, or any applicable method of the art. In some embodiments, the marking contains a lot number, expiration date, serial number, identifying information or any information or combination thereof. In some embodiments, the information is read, retrieved, or interpreted by an electrochemical analyzer during an interaction with the electrochemical sensor.

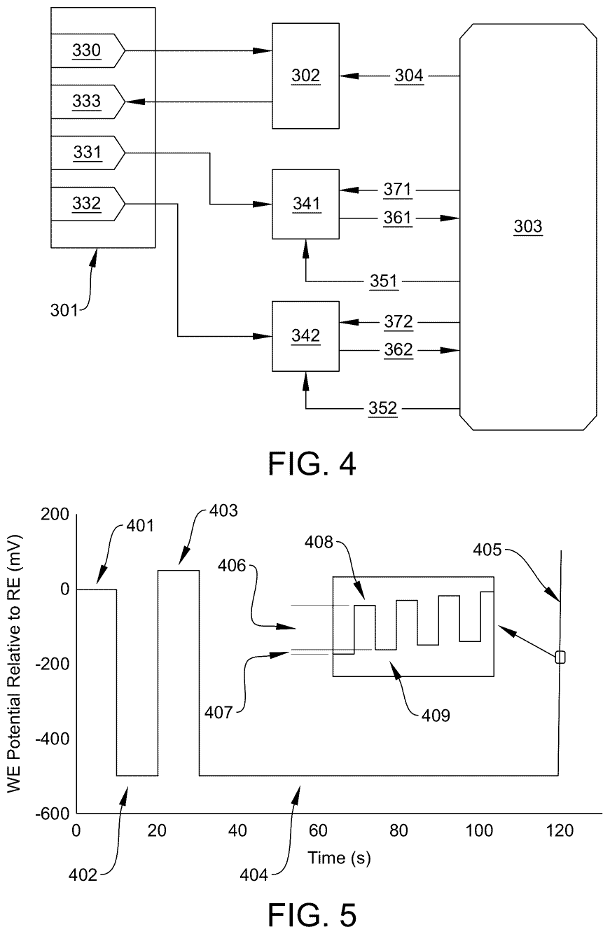

FIG. 4 depicts a schematic representation of a circuit of an embodiment of a microprocessor based electrochemical analyzer. Electrical connector 301 provides an electrical connection with an electrochemical sensor, such as sensor 200, connected to or inserted into the analyzer. In one embodiment, electrical connector 301 provides electrical contacts 330, 331, 332 and 333 making an electrical connection with an electrochemical. In one embodiment, the electrical contacts 330, 331, 332 and 333 are spring-loaded metallic contacts.

In one embodiment, electrical contact 330 connects with a reference electrode contact 230 connected to a reference electrode 220 of the electrochemical sensor 200, and the electrical contact 333 connects with the counter electrode contact 233 connected to the counter electrode 223 of the electrochemical sensor 200. The electrical contact 330 electrically transmits a potential or voltage of the reference electrode 220 to a potentiostat control unit 302. Potentiostat control unit 302 receives a reference electrode control signal 304 from a microprocessor 303 indicating a commanded reference electrode potential. Potentiostat control unit 302 automatically adjusts the voltage and current provided through the electrical contact 333 to the counter electrode 223 such that the potential of reference electrode 220 corresponds to that commanded by microprocessor 303.

In one embodiment, potentiostat control unit 302 includes an operational amplifier having a first input electrically connected to the reference electrode electrical contact 330, a second input electrically connected to the reference electrode control signal 304, and an output connected to the counter electrode electrical contact 333. In one embodiment, the reference electrode control signal 304 is a voltage provided by a digital to analog converter comprised in or controlled by the microprocessor 303.

In one embodiment, each of transimpedance amplifiers 341 and 342 comprises an operational amplifier having a first input electrically connected to a working electrode electrical contact, a second input electrically connected to a working electrode control signal, and an output electrically connected to, or feeding back into, the first input through a gain assembly. In one embodiment, the gain assembly comprises capacitors, resistors, switches, other components, or a combination thereof, assembled according to principles known in the art as to establish a relationship between a current flowing through the transimpedance amplifier and a voltage of the output, the voltage being provided to the microprocessor 303 for measurement.

In one embodiment, the electrical contacts 331 and 332 respectively connect with the first working electrode contact 231 and the second working electrode contact 232, themselves respectively connected with the first working electrode 221 and the second working electrode 222 of the electrochemical sensor 200. In one embodiment, the working electrode electrical contacts transmit electrical current flowing through the working electrodes to the transimpedance amplifiers 341 and 342. The transimpedance amplifiers automatically sink or source the current to maintain the voltage of working electrode contacts 331 and 332, and therefore of the working electrodes 221 and 222, at a constant value commanded by microprocessor 303 via working electrode control signals 351 and 352. In one embodiment, transimpedance amplifiers 341 and 342 further transmit voltages 361 and 362 related to the current flowing through working electrodes contacts 331 and 332 for measurement and analysis by microprocessor 303. In one embodiment, the relationship between the voltages 361 and 362 transmitted by each transimpedance amplifier and the current flowing through each working electrode is controlled by microprocessor 303 by way of gain control signals 371 and 372.

It is to be appreciated that the microprocessor 303 can include or interact with one or multiple systems or components, such as a controller, microcontroller, processor, microprocessor, memory, voltage source, current source, analog-to-digital converter, digital-to-analog converter, clock, sensor, transducer, thermometer or temperature sensor, display, touchscreen, user interface, software, firmware, barcode reader or any number, collection or assembly thereof.

In one embodiment, one or any number of control signals 304, 351, 352, 371 or 372 are delivered by the microprocessor 303 in a precisely timed sequence, the microprocessor 303 retrieving, gathering, reading or interpreting information describing the sequence from a memory such as a random access memory (RAM), a read-only memory (ROM) or any applicable memory described in the art. In one embodiment, a microprocessor based electrochemical analyzer provides means for information describing a sequence to be written to the memory.

In one embodiment, microprocessor unit 303 maintains working electrode control signals 351 and 352 at 0 (zero) volts, thereby causing the transimpedance amplifiers to maintain the working electrodes at 0 volts, and further varies reference electrode control signal 304, for example between −1 volt and +1 volt, as a function of time as required by an electrochemical measurement sequence being performed, such that the potential difference between each working electrode and the reference electrode is equal to reference electrode control signal 304 multiplied by −1. For example, were an electrochemical measurement require that working electrodes be maintained at a potential of −500 millivolts relative to, or 500 millivolts lower than, the reference electrode, reference electrode control signal 304 can be maintained at 500 millivolts, causing the potentiostat control unit to drive the counter electrode to ensure the reference electrode maintains a potential of 500 millivolts, and working electrode control signals 351 and 352 can be maintained at 0 volts, ensuring working electrodes are maintained at 0 volts, i.e. 500 millivolts lower than the reference electrode.

One skilled in the art will understand that the potentiostat system configuration illustrated in FIG. 4 is particularly suited to the operation of multiple working electrodes together acting on or measuring a same sample in an electrochemical sensor. More specifically, each working electrode can be provided with a transimpedance amplifier, which maintains its potential and measures its current independently of any other electrode, including other working, counter or reference electrode. While two working electrodes are schematically depicted, it is to be appreciated that any suitable number of, such as two, three, four, or more, working electrodes can be thus provided in an electrochemical sensor, connected to an electrical connector 301, and each measured independently by a dedicated transimpedance amplifier, while a potentiostat control unit independently controls the potential of the reference electrode, and the microprocessor monitors the relative potential of each working electrode relative to the reference electrode as a relationship, such as a difference, between each working electrode control signal and the reference electrode control signal 304.

FIG. 5 depicts an example of an electrochemical stripping assay, test or measurement sequence as may be performed by a microprocessor based electrochemical stripping analyzer. The assay can include a precisely timed sequence of potentials to be attained by a working electrode (WE) relative to a reference electrode (RE) in contact with a sample. A first step 401 in the sequence can be a delay step during which a potential of a WE is the same as, or 0 millivolts relative to, a RE potential for a relatively short duration such as approximately 0.1 second to 20 seconds. A second step 402 in the sequence can be a first preconditioning step during which a potential of a WE is lower than, such as approximately −500 millivolts relative to, a RE potential for a relatively short duration such as approximately 0.1 second to 20 seconds. A third step 403 in the sequence can be a second preconditioning step during which a potential of a WE is approximately equal to or higher than, such as approximately +50 millivolts relative to, an RE potential for a relatively short duration such as approximately 0.1 second to 20 seconds.

A fourth step 404 in the sequence can be a deposition step during which a potential of a WE is lower than, such as approximately −500 millivolts relative to, an RE potential for a relatively long duration such as approximately 10 to 300 seconds or more, the potentials and duration being selected to cause an electrochemical reduction, accumulation or deposition of one or multiple analytes or species of interest in a sample onto a WE.

A fifth step 405 in the sequence can be a stripping step during which a potential of a WE undergoes a ramp or scan in the anodic, increasing or positive direction relative to an RE potential, such as from a reduction or deposition potential to an oxidation or stripping potential of a species. The anodic scan can utilize the teachings of anodic stripping voltammetry, square wave voltammetry, square wave coulometry or any method of the art. In one embodiment, a stripping step includes a rapid sequence of forward steps 408 and reverse steps 409, each of a relatively short duration, such as 0.1 millisecond to 100 milliseconds or more, during which a potential of a WE is maintained constant relative to a RE potential. At the end of a forward step 408, the WE potential is decreased by an amount 406, such as 1 millivolt to 100 millivolts or more, to begin a reverse step 409. At the end of a reverse step 409, the WE potential is increased by a sum of an amount 406 and an increment 407, the increment being relatively small, such as 0.1 millivolt to 50 millivolts or more.

In one embodiment, during a step in the sequence, such as a delay, preconditioning, deposition or other step, a potential of a WE can be maintained at a constant value relative to an RE potential. In one embodiment, during a step in the sequence, such as a delay, preconditioning, deposition or other step, a potential of a WE relative to a RE potential can follow a square wave of a frequency between approximately 1 and 1000 hertz or more and an amplitude between approximately 0.1 millivolt and 100 millivolts or more, each period or cycle of the square wave including one forward step and one reverse step, the forward step following an increase in potential, and the reverse step following a decrease in potential.

In one embodiment, during a step in the sequence, a current flowing through a working electrode is measured at one or multiple time points, such as at the beginning, middle or end of the step; at regular intervals such as every second, every 0.1 second or every millisecond; at the end of every forward step and every reverse step of a square wave or stripping step; or any combination thereof. In one embodiment, currents flowing through each of multiple working electrodes are measured simultaneously or approximately simultaneously.

In some embodiments, a current measured during a forward step is called a forward current and a sequence or collection of forward currents is called a forward curve; a current measured during a reverse step is called a reverse current and a sequence or collection of reverse currents is called a reverse curve; a subtraction of a reverse current from a forward current is called a difference current, and a sequence or collection of difference currents, or a subtraction of a reverse curve from a forward curve, is called a difference curve.

It is to be appreciated that a electrochemical stripping assay, test or measurement sequence in accordance with the present disclosure can include any suitable number, sequence, or combination of delay steps 401, preconditioning steps 402 or 403, deposition steps 404 or stripping steps 405, or any other steps, each of any duration and potential, without departing from the scope of the present disclosure. For example an electrochemical measurement may include five preconditioning steps, a first stripping step, a deposition step, and a second stripping step.

In one example embodiment, the electrochemical measurement sequence can be altered after the sequence has begun, such as by increasing or decreasing a duration, potential or other characteristic of a step of the sequence, or by changing a number, order or selection of steps, for example in response to a result or value of a measurement or computation made during the sequence.

In one embodiment, an electrochemical stripping assay, test or measurement sequence such as depicted in FIG. 5. is conducted on an electrochemical stripping sensor such as depicted in FIGS. 2-3 that is inserted into a microprocessor based electrochemical analyzer such as depicted in FIG. 4., whereby the microprocessor 303 commands transimpedance amplifiers 341 and 342 by way of control signals 351 and 352 to maintain working electrodes 221 and 222 at a potential of 0 volt, and commands the potentiostat control unit 302 by way of a precisely timed sequence of control signal 304 to adjust a voltage and current provided to the counter electrode 323 such that a potential of the reference electrode 220, transmitted to the potentiostat control unit 302 by way of the reference electrode contact 230, is of equal value and opposite sign as the desired WE potential relative to RE.

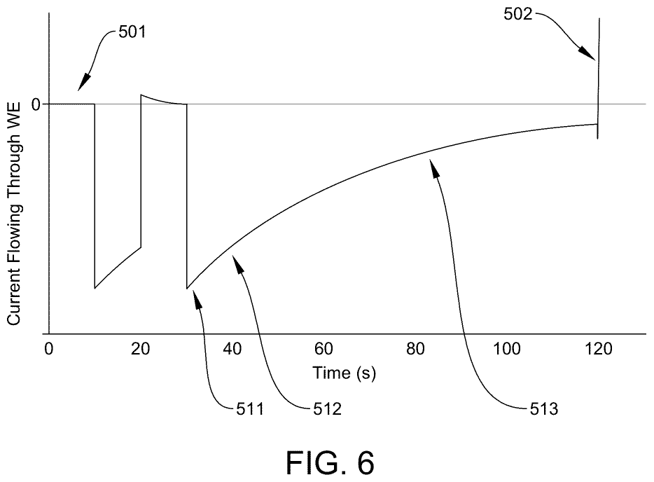

FIG. 6 depicts a current flowing through a working electrode during an electrochemical measurement sequence such as that depicted in FIG. 5. A working electrode current 501 can vary throughout the measurement sequence as a function of time, potential, sample composition, or other factors. For example, during a deposition step 404, a current flowing through a working electrode can be far from zero at a first or initial moment 511, rapidly trend towards zero at a second moment 512, and continue converging more slowly towards zero through a third moment 513, until the end of the deposition step 404 and the beginning of a stripping step 405 producing increasing working electrode current through moment 502.

In one embodiment, a microprocessor of an electrochemical analyzer can perform measurements of a working electrode current at two, three or more moments during at one or multiple steps of an electrochemical measurement sequence, such as a delay, preconditioning, deposition or stripping step, and compare the measurements, or results of computations performed thereon, to a range of acceptable or expected values stored in a memory. In one embodiment, the microprocessor can further use the result of the comparison to alter, modify, correct, compensate, qualify, reject or process in any other way a measurement outcome.

In one embodiment, the computations performed on measurements can include addition, subtraction, multiplication, division, averaging, differentiation, integration or any number, sequence or combination thereof. In one embodiment, the computations involve fitting a parametric curve, such as a linear, quadratic, polynomial, exponential or logarithmic curve or any combination thereof, to the measurements using methods known in the art, such as ordinary least squares, linear or nonlinear regression algorithms, and the resulting best fit parameters compared to ranges of acceptable or expected values.

For example, the microprocessor can make a first current measurement at a first moment 511 of a deposition step and at second current measurement at a second moment 512 of the deposition step, the second moment being between approximately 1 and 20 seconds or more later than the first moment; compute the rate of change of the current, for example by subtracting the second measurement from the first measurement and dividing by a duration elapsed between the first moment and the second moment; and compare the rate of change to a range of acceptable values stored in a memory. In one embodiment, if the rate of change is outside the range of acceptable values, the microprocessor may stop, shorten, lengthen or alter an electrochemical measurement sequence or step thereof, apply a compensation or correction to an outcome of the electrochemical measurement, for example by way of mathematical operations or computations, identify the outcome as invalid, or take any other action or combination thereof.

As a further example, the microprocessor may make current measurements at multiple moments, such as approximately 2, 3, 10, 20, 100, 1000 or more moments, of a deposition step to obtain a current curve; fit to the current curve a parametric curve such as an exponential curve using computations such as a nonlinear regression algorithm; compute a deviation or difference between a current measurement and the fitted parametric curve; compare the deviation to a range of acceptable values stored in a memory; and identify as invalid an outcome of an electrochemical measurement. As a further example, the microprocessor can compute a ratio between a first and a second value, the first value being a value of a parameter of the parametric curve fitted to the current curve, and the second value being a square wave coulometry result; compare the ratio to a range of acceptable values stored in a memory; and apply a compensation or correction to an outcome of the electrochemical measurement.

In some embodiments, a measurement or computation, such as rate of change of a current or a value of a parameter of a parametric curve fitted to a current curve, in a step of an electrochemical measurement sequence, such as a deposition step, is indicative of an interference from a species present in a sample, such as reduced glutathione (GSH), molecules that contain sulfhydryl groups, or molecules that bind to gold or metallic surfaces or colloids. For example, a lower rate of change of a current during a deposition step may be indicative of an interference from GSH in a sample. In some embodiments, a range of acceptable values of the rate of change or parameter is defined to exclude values, such as lower or higher values, indicative of an interference in a sample.

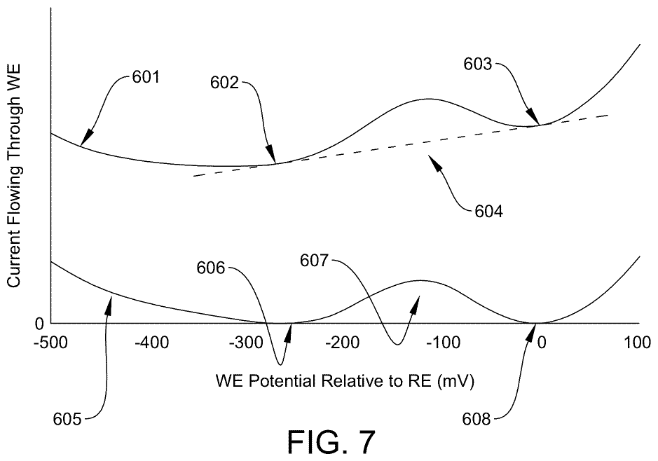

FIG. 7 depicts a computation of a square wave coulometric (SWC) value, result or stripping signal from an electrochemical measurement sequence such as can be performed by a microprocessor based electrochemical analyzer. A difference curve 601 obtained during a stripping step 405 of an electrochemical measurement sequence and is plotted against a potential of a working electrode relative to a reference electrode. A straight line or baseline 604 is computed by methods known in the art so as to be tangent to difference curve 601 at a first point 602 and at a second point 603, the first point being at a WE potential lower than that of a stripping peak of an analyte or species, and the second point being at a WE potential higher than that of the stripping peak of the analyte.

The baseline is then subtracted from the difference curve to obtain a corrected curve 605 whose current value substantially equals zero at a first minimum 606 and at a second minimum 608, the first minimum being at a WE potential lower than that of a stripping peak of an analyte or species, and the second minimum being at a WE potential higher than that of the stripping peak of the analyte.

A SWC result or stripping signal is a working electrode current measurement which can be computed as an area 607 of a stripping peak of corrected curve 605 located between first minimum 606 and second minimum 608. For example, a corrected curve 605 can be numerically integrated between first minimum 606 and second minimum 608 to obtain the SWC result. A SWC result or stripping signal can be computed as a height, such as a maximum height, of a stripping peak of corrected curve 605. In some embodiments, a SWC result can be expressed as a unitless quantity.

In one embodiment, a microprocessor further processes a SWC result to obtain a value of a concentration, quantity or amount of an analyte or a species in a sample, for example in units of microgram of analyte per deciliter of sample (μg/dL), micromolar of analyte per liter of sample (μmol/L), parts per million (ppm) or parts per billion (ppb). For example, a SWC result can be added or subtracted to, or combined or averaged with, another SWC result, a measurement or rate of change of a current of a deposition step, to obtain a new or corrected SWC result. As a further example, a SWC result or corrected SWC result can be multiplied by a factor dependent on any value placed in a memory of a microprocessor, such as a temperature measurement made by a thermometer interfaced with the microprocessor to obtain a further corrected SWC result. As a further example, a SWC result or corrected SWC result can be inputted into a calibration function, equation, operation, table, algorithm or a combination thereof, to obtain a value of a concentration of an analyte, such as lead, in a sample, such as blood, in relevant or clinically recognized units, such as μg/dL. In one embodiment, a calibration function utilizes calibration data stored in a memory, the calibration data having been written into the memory by means provided by the microprocessor.

In one embodiment, a microprocessor can compute a first SWC result or concentration of an analyte from a first difference curve representing a current of a first working electrode; compute a second SWC result or concentration of an analyte from a second difference curve representing a current of a second working electrode; compute a difference or ratio between the first SWC result or concentration and the second SWC result or concentration; compare the difference or ratio to a range of acceptable values in a memory; and use the result of the comparison to alter, modify, correct, compensate, qualify, reject or process in any other way a measurement outcome. As an example, a microprocessor can compare blood lead concentration values obtained from two working electrodes of a sensor measuring a sample, determine whether both concentration values are within a certain percentage of each other, such as 1%, 3%, 10%, or within a certain margin of each other, such as approximately 0.1 μg/dL, 0.5 μg/dL, 1 μg/dL, 2 μg/dL or more, and if they are not, identify the electrochemical measurement result as invalid.

In some embodiments, the potentiostat circuitry provided herein applies to a counter electrode whatever electrical potential and current is necessary for a reference electrode to adopt a commanded potential. In some embodiments, one or two or more working electrodes can each be maintained at a potential, such as 0 (zero) volt, by way of being electrically connected to one or many transimpedance amplifiers. This electrical connection can be achieved by traces, wires, contacts or other elements made of electrically conductive materials such as inks or resins containing particles of carbon or metal, spring-loaded metallic pins, printed circuit board traces, insulated wires, solder or other means or combination thereof. This transimpedance amplifier can include in an operational amplifier having a first input to which a working electrode is electrically connected, a second input to which a potential or control signal is applied by a potentiostat controller or other element, and an output which is fed back to the first input connected to the working electrode through one or a multitude of resistor, capacitor, switches or other electronic components or a combination thereof. The output voltage can be measured by way of methods described in the art as an indication of an electrical current flowing through the working electrode connected to the first input.

The difference in potential between a working electrode and reference electrode can be considered as a driver of the chemical reactions expected to occur at the working electrode, such as the reduction or oxidation of ions or species, including metals such as lead. As may be understood by those versed in the art, the difference in potential between a working electrode and reference electrode may be referred to as the “working electrode potential” or variations thereof for brevity, but such terminology may also refer to the actual potential at which the working electrode is being maintained relative to another potential reference level of the potentiostat, which can be referred to as ground, 0 or zero volt, Vref or some other term.

The actual potential at which each working electrode is maintained by a transimpedance amplifier can be constant throughout the electrochemical measurement or can be varied as a function of one or a combination of time, potential, or current, during the electrochemical measurement. This constant or varying potential can be achieved by way of a control signal sent to a transimpedance amplifier to which one or multiple working electrodes are connected.

In accordance with the present disclosure, many or all working electrodes can be maintained at the same potential by being connected to a single transimpedance amplifier. In this case, the current measured by the transimpedance amplifier can be understood to represent the sum total of current flowing through all working electrodes connected to it. The approach can be utilized to increase the effective working electrode area where each working electrode's shape, size, or combination thereof is otherwise constrained due to limitations of manufacturing processes or other considerations, such as the deposition, spraying, drying, activation, inspection or other operations performed on working electrode surfaces, coatings, chemistries or a combination thereof. It may be desirable to increase the effective working electrode area to increase the number of molecules reacting during an electrochemical measurement, increase the resulting current flowing through a transimpedance amplifier, increase the signal produced thereby, increase the sensitivity to low levels of analyte such as lead in biological samples, or a combination thereof.

Many or all working electrodes can be maintained at the same potential by each being solely connected to a dedicated transimpedance amplifier, each transimpedance amplifier being provided with the same control signal. In this case, the current measured by each transimpedance amplifier can be understood to represent the current flowing through the sole working electrode connected to it. The different values of current measured for each working electrode can therefore by compared, added, subtracted, multiplied, averaged or otherwise processed by a controller or similar system of the art. Through such comparison and processing, one can obtain the benefits of an increased effective working electrode area described previously by, for example, adding or averaging the signals from each transimpedance amplifier. Furthermore, additional or complementary processing can yield information on the quality or other characteristics of each working electrode and on the quality of the resulting signal.

For example, one can compare the measured current flowing through a first working electrode to the current flowing through a second working electrode substantially similar to the first in geometry, materials, coatings, surface chemistry and construction, at one or many moments during the electrochemical measurement, to detect any discrepancy between the two electrodes that could indicate fabrication defects or other undesirable or noteworthy conditions. As a specific example, a measurement of the current flowing through a first working electrode can be subtracted from a measurement of the current flowing through a second working electrode substantially identical to the first to yield a current difference, which is then compared to a range of expected or acceptable values, such comparison further triggering electrochemical measurements performed by either or both working electrodes to be corrected by further processing or rejected as unreliable.

As a further example, one can compare the measured current flowing through a first working electrode to the current flowing through a second working electrode differing from the first in geometry, materials, coatings, surface chemistry or construction, or a combination thereof, at one or many moments during the electrochemical measurement, and make any determination from their relationship as can be of use for the purposes of rejecting or correcting the electrochemical measurement due to the effects for interferences introduced by chemical species, temperature, electromagnetic fields, or other sources. As a specific example, a measurement of a background current flowing through a first working electrode can be subtracted from a measurement of an analytical current flowing through a second working electrode substantially identical to the first except for the addition of a compound or feature enhancing its reactivity to a chemical species of interest, such as lead, to obtain a background-corrected analytical measurement having desirable characteristics relative to a non-background-corrected measurement.

Such methods of measurement, processing, subtraction, comparison, and determination of acceptability in accordance with the present disclosure can be performed in any order by digital computers, microprocessors, controllers, memory, software or a combination thereof, on any number of working electrodes or groups thereof, at any single or multiple time points during the electrochemical measurement, to achieve a desirable result.

As a specific example, the current flowing through each of two working electrodes while the reference electrode potential is maintained at a constant value can be measured at two, three or more moments in time. At each of these moments, the current flowing through the first electrode can be compared to the current flowing through the second electrode to ensure they are within a certain acceptable range of each other after which further analysis is allowed to proceed. Then, for each electrode, the current at a first moment is subtracted from the current measured at a second moment to determine the rate of increase or decrease of the current between the first and second moments which rate is compared to a range of acceptable or expected values. Alternatively, a parametric curve can be fitted to current measurements made at multiple time points using methods known in the art, such as ordinary least squares, linear or nonlinear regression algorithms, and the resulting best fit parameters compared to ranges of acceptable or expected values.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto.

We claim:

1. A system for measuring lead concentration in blood, comprising:
a housing substantially enclosing a data processing system comprising a power supply, a microprocessor, and a memory;
an electrochemical sensor insertable in the housing and comprising a first working electrode, a second working electrode, a reference electrode and a counter electrode, each adapted to be in contact with a sample of a blood, wherein each of the first working electrode, the second working electrode, the reference electrode, and the counter electrode are printed and wherein each of the first working electrode and the second working electrode comprise a metallic colloid coating
an electrical connector adapted to couple signal output from the inserted electrochemical sensor with the data processing system;
circuitry within the housing configured to apply a square wave coulometric analytical technique to the electrochemical sensor;
a source of potential within the housing adapted to apply a potential to the circuitry; and wherein the microprocessor is programmed to apply the potential to the circuitry to measure the signal output from the electrochemical sensor at a plurality of different times to determine the amount of the analyte in the electrolyte.

2. The system of claim 1, wherein the metallic colloid comprises any of a gold colloid and a platinum colloid.

3. The system of claim 1, wherein each of the first working electrode and second working electrode comprises a layer of conductive ink, a layer of ink containing carbon particles, a layer of glassy carbon particles or nanoparticles, a layer of cationic polymer, and a layer of the metallic colloid.

4. The system of claim 1, wherein the source of potential comprises a first transimpedance amplifier configured to electrically couple to the first working electrode and a second transimpedance amplifier configured to electrically couple to the second working electrode.

5. The system of claim 4, wherein the microprocessor is programmed to measure a first current measured for the first working electrode and a current measured for the second working electrode to detect discrepancies between the measured currents.

6. The system of claim 4, wherein the microprocessor is programmed to process a first current measured for the first working electrode and a second current measured for the second working electrode, wherein the processing is any of a comparison process, an addition process, a subtraction process, a multiplication process, and an averaging process.

7. The system of claim 1, wherein at each of the plurality of different times, the first current measured for the first working electrode is compared to the second current measured for the second working electrode to determine whether the first and second currents are within a threshold range of each other.

8. The system of claim 1, wherein the first current measured for the first working electrode at a first of the plurality of times is subtracted from the first current measured for the first working electrode at a second of the plurality of times to determine whether a rate of change is within a threshold range.

9. The system of claim 8, wherein the second current measured for the second working electrode at the first of the plurality of times is subtracted from the second current measured for the second working electrode at the second of the plurality of times to determine whether a rate of change is within a threshold range.

10. The system of claim 1, wherein the plurality of different times comprises at least three different times.

11. The system of claim 1, wherein a combined surface area of the first working electrode and the second working electrode is greater than a surface area of the reference electrode.

12. An electrochemical stripping sensor, comprising:
a body defining a fluidic channel and a sample inlet port, wherein the sample inlet port is in fluid communication with the fluidic channel;
a plurality of electrodes positioned within the fluidic channel and adapted to be in contact with a sample of a blood, wherein the plurality of electrodes comprises a reference electrode, a counter electrode, and a plurality of printed working electrodes, wherein each of the plurality of sprinted working electrodes comprise a metallic colloid coating; and
a plurality of contacts, wherein each of the plurality of contacts is in electrical communication with a respective one of the plurality of electrodes.

13. The electrochemical stripping sensor of claim 12, wherein the plurality of printed working electrodes comprises a first working electrode and a second working electrode.

14. The electrochemical stripping sensor of claim 13, wherein the plurality of contacts comprises a first working electrode contact, a second working electrode contact, a reference electrode contact, and a counter electrode contact.

15. The electrochemical stripping sensor of claim 13, wherein a configuration of the first working electrode is the same as a configuration of the second working electrode.

16. The electrochemical stripping sensor of claim 13, wherein a configuration of the first working electrode differs from a configuration of the second working electrode in any of size, shape, and material.

17. The electrochemical stripping sensor of claim 12, wherein the body comprises a base layer having an upper surface and a channel layer coupled to the upper surface of the base layer.

18. The electrochemical stripping sensor of claim 17, the channel layer at least partially defines the fluidic channel, wherein the reference electrode, the plurality of printed working electrodes, and the counter electrode are linearly arranged along the fluidic channel.

19. The electrochemical stripping sensor of claim 13, wherein the reference electrode comprises conductive silver ink, the first working electrode and the second working electrode each comprise carbon conductive ink onto which is applied a layer of gold colloid suspended in a cationic polymer, and wherein the counter electrode comprises silver conductive ink onto which is applied a layer of carbon conductive ink.

20. A system for measuring the amount of an analyte in a blood which comprises:
a housing substantially enclosing a data processing system comprising a power supply, a microprocessor, and a memory;
an electrochemical sensor insertable in the housing, wherein the electrochemical sensor comprises
a body defining a fluidic channel and a sample inlet port in fluid communication with the fluidic channel;
a plurality of electrodes positioned within the fluidic channel and adapted to be in contact with a sample of a blood, wherein the plurality of electrodes comprises a reference electrode, a counter electrode, a first printed working electrode, and a printed second working electrode; wherein the reference electrode comprises conductive silver ink, the first printed working electrode and the second printed working electrode each comprise carbon conductive ink onto which is applied a layer of gold colloid suspended in a cationic polymer, and wherein the counter electrode comprises silver conductive ink onto which is applied a layer of carbon conductive ink; and
a plurality of contacts, wherein each of the plurality of contacts is in electrical communication with a respective one of the plurality of electrodes;
an electrical connector adapted to couple signal output from the inserted electrochemical sensor with the data processing system;
circuitry within the housing configured to apply a square wave coulometric analytical technique to the electrochemical sensor; and
a source of potential within the housing adapted to apply a potential to the circuitry; and
wherein the microprocessor is programmed to apply the potential to the circuitry to measure the signal output from the electrochemical sensor at a plurality of different times to determine the amount of the analyte in the electrolyte.
